(19) United States
(12) Patent Application Publication
(10) Pub. No.: US 2021/0393965 A1
(43) Pub. Date: Dec. 23, 2021

(54) A SYSTEM, A METHOD, A COMPUTER PROGRAM PRODUCT AND AN ELECTRIC STIMULATION SIGNAL

(71) Applicant: Erasmus University Medical Center Rotterdam, Rotterdam (NL)

(72) Inventors: Biswadjiet Sanjay HARHANGI, Rotterdam (NL); Franciscus Johannes Petrus Maria HUYGEN, Rotterdam (NL)

(73) Assignee: Erasmus University Medical Center Rotterdam, Rotterdam (NL)

(21) Appl. No.: 17/292,916

(22) PCT Filed: Nov. 12, 2019
(86) PCT No.: PCT/NL2019/050737

§ 371 Date: May 11, 2021

(30) Foreign Application Priority Data
Nov. 15, 2018 (NL) 2022004

(51) Int. Cl. A61N 1/36; A61N 1/05

(52) U.S. Cl. CPC A61N 1/36139; A61N 1/0551; A61N 1/36067; A61N 1/37247; A61N 1/36171; A61N 1/36175; A61N 1/36132

(57) ABSTRACT

The invention relates to a system for preventing and/or reducing muscle spasms and improving postural stability in a patient suffering from muscle spasm and postural instability. The system comprises an electrode configured to be positioned to stimulate a target dorsal root ganglion associated with the muscle spasm and postural instability. The system also comprises a pulse generator provided with an output port connectable to the electrode. The pulse generator includes a processor configured to generate an electric stimulation signal at the electrode while connected. Here, the electric stimulation signal stimulates the dorsal root ganglion with an electric pulse while so positioned. Further, the system comprises a user interface configured to set at least one user-specified parameter of the electric pulse.

The invention relates to a system for treating a patient with movement disorder.

Patent publication EP 2 670 478 B1 discloses a system for treating a patient having a movement disorder, the system comprising electronic circuitry providing a stimulation signal having an electric pulse stimulating a portion of a target dorsal root ganglion via an electrode, in order to reduce a symptom of the movement disorder.

It is an object of the present invention to provide an improved system for treating a patient. Thereto, according to the invention, a system is provided for preventing and/or reducing muscle spasms and improving postural stability in a patient suffering from muscle spasms and postural instability, the system comprising:
an electrode configured to be positioned to stimulate a target dorsal root ganglion associated with the muscle spasms and postural instability;
a pulse generator provided with an output port connectable to the electrode,
wherein the pulse generator includes a processor configured to generate an electric stimulation signal at the electrode while connected, the electric stimulation signal stimulating the dorsal root ganglion with an electric pulse while so positioned, and wherein the system further comprises a user interface configured to set at least one user-specified parameter of the electric pulse.

The invention is at least partially based on the surprising observation that both muscle spasms can be prevented and postural stability can be improved by setting at least a parameter value of a stimulating electric pulse in a specific range. It has been found, more specifically, that the combined effect may occur when at least an amplitude, a frequency or a pulse width of the electric pulse is set to a value in a pre-defined range of values. Preferably, the amplitude may be chosen in a range from circa 0.01 mA to circa 2 mA, the frequency may be chosen in a range from circa 1 Hz to circa 100 Hz, and/or the pulse width of the electric pulse may be chosen in a range from circa 10 microseconds to circa 500 microseconds. More preferably, the amplitude, the frequency and the pulse width are selected from a value in the above mentioned value ranges, respectively, in combination.

By providing a user interface enabling a user to set at least one user-specified parameter of the electric pulse, the user is facilitated in selecting the at least one user-specified parameter in the desired regime, preferably the three user-specified parameters of the electric pulse, viz. the amplitude, the frequency and the pulse width, in combination, then obtaining the unexpected finding that muscle spasms are prevented and postural stability is improved.

The invention also relates to a method.

Further, the invention relates to a computer program product. A computer program product may comprise a set of computer executable instructions stored on a data carrier, such as a CD or a DVD. The set of computer executable instructions, which allow a programmable computer to carry out the method as defined above, may also be available for downloading from a remote server, for example via the Internet, e.g. as an app.

Also, the invention relates to an electric stimulation signal.

Further advantageous embodiments according to the invention are described in the following claims.

It should be noted that the technical features described above or below may each on its own be embodied in a system or method, i.e. isolated from the context in which it is described, separate from other features, or in combination with only a number of the other features described in the context in which it is disclosed. Each of these features may further be combined with any other feature disclosed, in any combination.

The invention will now be further elucidated on the basis of a number of exemplary embodiments and an accompanying drawing. In the drawing:

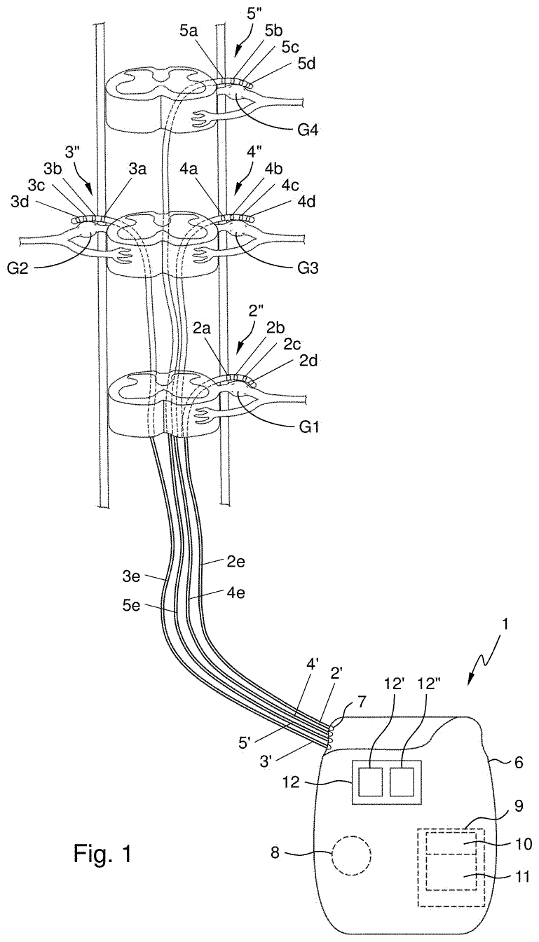

FIG. 1 shows a schematic view of a system according to the invention for preventing and/or reducing muscle spasm and improving postural stability in a patient suffering from muscle spasm and postural instability;

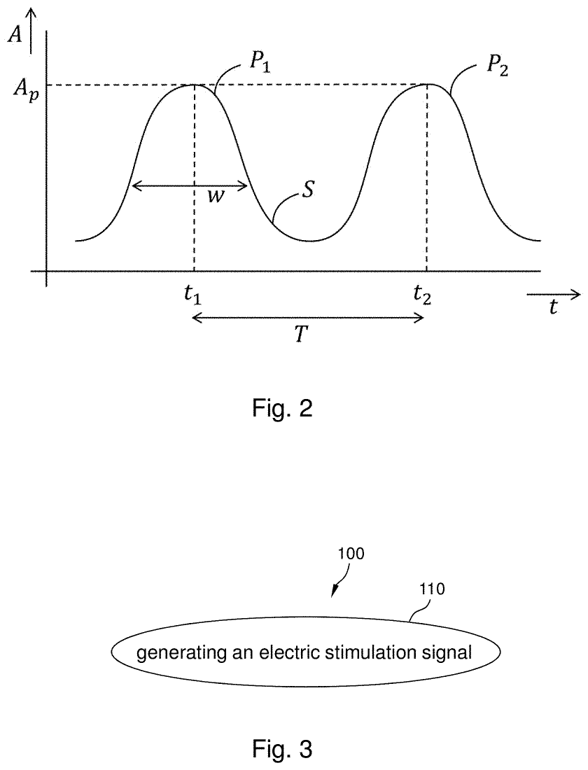

FIG. 2 shows an electric stimulation signal according to the invention for preventing and/or reducing muscle spasms and improving postural stability in a patient suffering from muscle spasm and postural instability;

FIG. 3 shows a flow chart of a method according to the invention;

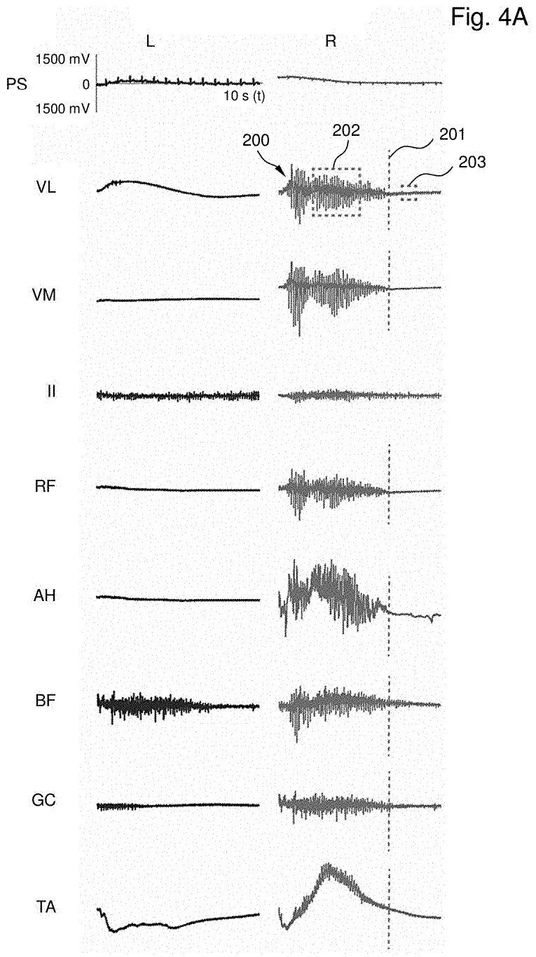

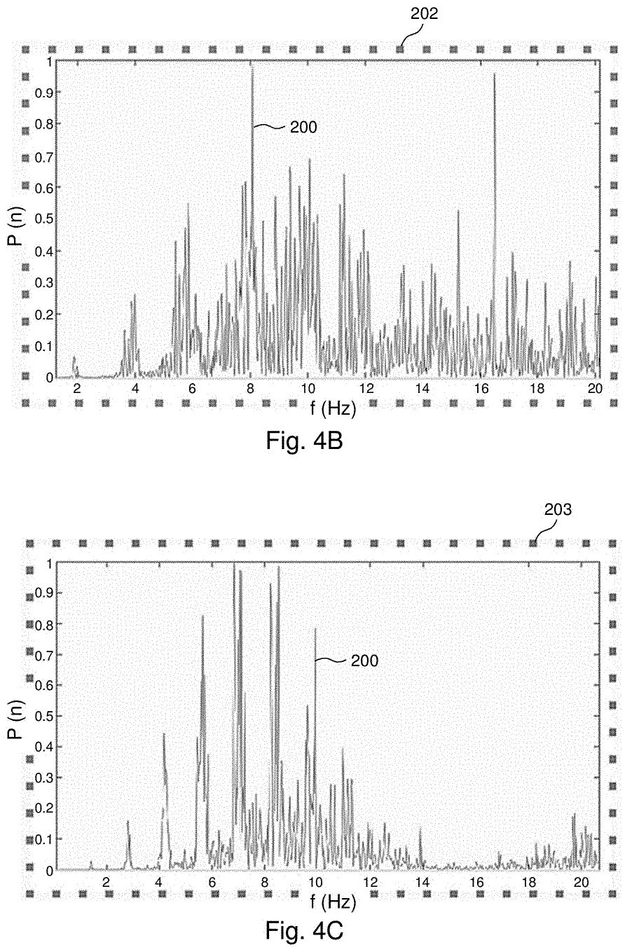

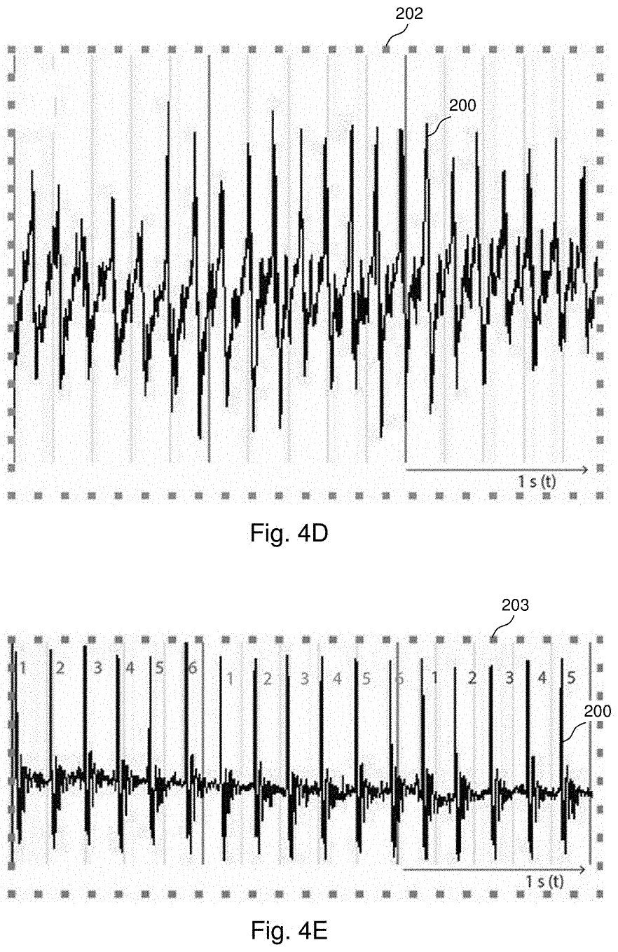

FIG. 4A shows electromyography traces of bilateral muscle groups of a patient;

FIG. 4B shows a frequency spectrum of a Vastus Lateralis electromyography trace prior to application of an electric stimulation signal;

FIG. 4C shows a frequency spectrum of the Vastus Lateralis electromyography trace after application of an electric stimulation signal;

FIG. 4D shows the Vastus Lateralis electromyography trace as a function of time prior to application of an electric stimulation signal, and

FIG. 4E shows the Vastus Lateralis electromyography trace as a function of time after application of an electric stimulation signal.

It is noted that the figures show merely preferred embodiments according to the invention. In the figures, the same reference numbers refer to equal or corresponding parts.

FIG. 1 shows a schematic view of a system 1 according to the invention. The system 1 is arranged for preventing and/or reducing muscle spasm and improving postural stability in a patient suffering from muscle spasm and postural instability. The system 1 has a multiple number of sets of electrodes, each set of electrodes 2, 3, 4, 5 having a multiple number of electrodes 2a-d, 3a-d, 4a-d, 5a-d. In the embodiment shown in FIG. 1, the electrodes of each set 2, 3, 4, 5 are bundled in a string of electrodes that is preferably sealed by a sleeve 2e, 3e, 4e, 5e. The system 1 also has a pulse generator 6 provided with output ports connectable to proximal ends 2′, 3′, 4′, 5′ of the electrodes. Optionally, the pulse generator 6 is implantable. Further, the system is provided with a user interface 12.

In the embodiment shown in FIG. 1, distal ends 2″, 3″, 4″, 5″ of the electrodes of each set 2, 3, 4, 5 are positioned at a target dorsal root ganglion DRG associated with the muscle spasm and postural instability of the patient. As an example, the distal ends of the electrodes can be positioned at the targeted dorsal root ganglion like L1, L2, L3, L4, L5, S1, S2, S3, S4, S5, Thoracic or Cervical levels.

In the shown embodiment, distal ends of the electrodes 2a-d of a first set 2 are located at a first dorsal root ganglion G1, at a first level, while distal ends of electrodes 3a-d, 4a-d of a second and third set 3, 4, respectively, are located at a second and third dorsal root ganglion G2, G3, respectively, at a common, second level. Further, distal ends electrodes 5a-d of a fourth set 5 are located at a fourth dorsal root ganglion G4, at a third level.

It is noted that the system may include more than four sets of electrodes, e.g. five, six, seven, eight or even more sets of electrodes such as ten or twenty sets of electrodes, or less than four sets, e.g. three, two or one set of electrodes. Further, the number of electrodes in each set may be two, three, four, five, six, seven, eight or even more, e.g. ten or twenty. Also, single electrodes can be provided, not assembled together in a string or lead, but separately. Typically, at least two electrodes are located at a target dorsal root ganglion DRG for providing an electrical stimulation signal.

Generally, the electrodes are configured to be positioned to stimulate, at their distal ends, a target dorsal root ganglion DRG associated with the spinal cord injury of the patient. For positioning the electrodes, each string of electrodes 2, 3, 4, 5 can be individually advanced with a spinal column, e.g. in an antegrade direction, and subsequently guided towards the respective target dorsal root ganglion DRG such that the distal ends 2″, 3″, 4″, 5″ are located proximate to the target dorsal root ganglion DRG G1, G2, G3, G4. Further, in principle, each electrode of a set of electrodes 2, 3, 4, 5 may be able to selectively stimulate the target dorsal root ganglion DRG by selecting pulse parameters, electric field flux, electrode configuration, electrode position, electrode orientation and/or electrode shape.

The pulse generator 6 is provided with a multiple number of output ports 7 connectable to the respective electrodes 2a-d, 3a-d, 4a-d, 5a-d. In principle, the electrodes can be connected and disconnected from the output ports 7 of the pulse generator 6. Further, the pulse generator 6 includes a power supply 8 and a control unit 9 provided with a processor 10 and a memory 11.

The power supply 8 may include a battery or mains connector for feeding the system 1 with power.

The processor 10 is configured to generate an electric stimulation signal S at each of the electrodes 2a-d, 3a-d, 4a-d, 5a-d while connected, the electric stimulation signal S stimulating the dorsal root ganglion DRG with an electric pulse P while so positioned. The memory 11 of the control unit 9 is configured to store data, such as parameters of the electric stimulation signal S or the electric pulse P of the signal S. Optionally, the control unit 9 may include additional components, e.g. for facilitating server or Internet connectivity.

The user interface 12 is configured to set at least one user-specified parameter of the electric pulse P. As an example, the user interface 12 may include a single or a multiple number of user operable elements 12′ such as switches or buttons such that a user may input a single or a multiple number of user-specified parameters, e.g. an amplitude, frequency and/or pulse width of the electric pulse P. Further, the user interface 12 may include another or an additional interaction mechanism for allowing a user to input a parameter value to the user interface 12, e.g. using speech or digital input e.g. using a removable memory card. Further, the user interface 9 may include a feed back module 12″ for feeding back a parameter value that is input by the user, such as a display.

The user interface 12 may be integrated with the pulse generator 6, e.g. in a single casing also including the power supply 8 and the control unit 9 as shown in FIG. 1. Alternatively, the user interface 12 may be implemented as a separate device communicating with the pulse generator 6 via a wired or wireless data connection, e.g. if the pulse generator 6 is implanted in the body of the patient.

Advantageously, the user interface 12 may be configured to set the at least one user-specified parameter of the electric pulse P only to a value in a pre-specified range. Then, a user of the system 1 may be supported in selecting a proper parameter value enabling the electric stimulation signal S to prevent muscle spasm and improve postural stability in the patient suffering from a spinal cord injury. Alternatively, the user may select a parameter value outside beyond a pre-specified range. Optionally, a warning signal may then be generated, e.g. an audible or visible signal. Further, in another embodiment, no parameter value range may be pre-specified. In yet a further embodiment, the user may modify or set a parameter value range.

Generally, the user interface 12 may be configured to set multiple user-specified parameters of the electric pulse P only to a respective value in a respective pre-specified range, e.g. an amplitude value within a pre-specified amplitude range, a frequency value within a pre-specified frequency range, and a pulse width value within a pre-specified pulse width range.

As an example, the at least one user-specified parameter includes a parameter representing an amplitude A of the electric pulse P, such that the amplitude can be selected within a range between circa 0.01 mA and circa 2 mA. Specifically, the amplitude A can e.g. be set to circa 0.025 mA, circa 0.05 mA, circa 0.075 mA, circa 0.1 mA, circa 0.2 mA, circa 0.5 mA, circa 1 mA, or circa 1.5 mA. However, in principle, also an amplitude A beyond the above range can be selected, e.g. circa 0.005 mA or circa 4 mA. As a further example, the at least one user-specified parameter includes a parameter representing a repetition frequency f of the electric pulse P, such that the frequency can be selected within a range between circa 1 Hz and circa 100 Hz. Specifically, the frequency can e.g. be set to circa 2 Hz, circa 4 Hz, circa 10 Hz, circa 20 Hz, circa 30 Hz, circa 40 Hz, circa 50 Hz, circa 60 Hz, circa 70 Hz, circa 80 Hz or circa 90 Hz. However, as indicated above, also a frequency outside the pre-selected range can be selected, e.g. circa 0.5 Hz or circa 150 Hz. As an alternative to a repetition frequency, a time period of a single cycle of the stimulation signal S can be set. As another example, the at least one user-specified parameter includes a parameter representing a pulse width w of the electric pulse P, such that the pulse width can be selected within a range between circa 10 microseconds and circa 500 microseconds. Specifically, the pulse width can be set to circa 20 microseconds, circa 50 microseconds, circa 100 microseconds, circa 200 microseconds or circa 400 microseconds. However, as indicated above, also a pulse width w outside the pre-selected range can be selected, e.g. circa 2 or circa 5 milliseconds.

Also other or corresponding parameters of the electric stimulation signal S can be user specified such as a duty cycle, a pulse slope, a pulse shape, a pulse energy and/or a pulse voltage. As an example, the pulse may have a sinusoidal shape, a block shape, a triangular shape, a trapezoidal shape or another shape such as semi-circular shape. In principle, a single user-specified parameter can be set by a user interacting with the user interface 12, e.g. the frequency f of the electric pulse P. Further, a multiple number of user-specified parameters can be set by the user, e.g. the frequency f and the amplitude A of the electric pulse P, or the frequency f, the amplitude A and the pulse width w of the electric pulse P.

Optionally, the at least one user-specified parameter also includes a parameter setting a sequence of high frequency electric pulses P preceding, following or alternating electric pulses P having a repetition frequency in the range between circa 1 Hz and circa 100 Hz. Here, the high frequency electric pulses P have a frequency above circa 100 Hz. As an example, a so-called burst of high frequency pulses P can be generated prior to or after a series of regular electric pulses P in a normal, low frequency regime, i.e. not exceeding circa 100 Hz. Also, the high frequency pulses P can be provided between two series of electric pulses P in the normal, low frequency regime. The high frequency pulses may have a frequency that is significantly higher than the upper bound of the low frequency range, i.e. significantly higher than circa 100 Hz, e.g. circa 300 Hz, circa 500 Hz or circa 700 Hz, or even higher, e.g. circa 10 kHz. Further, the high frequency pulses may have multiple spectral components above the normal, low frequency regime. As an example, the high frequency pulses may include 500 Hz and 700 Hz pulses. As another example, the high frequency pulses may include a small band or broad band spectrum components, such as a noise signal. The sequence of high frequency pulses may have a duration of several milliseconds, or less or more than several milliseconds. Further, a series of high frequency pulses, e.g. 10 kHz pulses, may be applied that is not preceding or following a low frequency series of electric pulses. Generally, a user-specified parameter may define a single or multiple interval lengths of pulses.

In principle, a single user-specified parameter can be set by a user interacting with the user interface 12, e.g. the frequency f of the electric pulse P. Further, a multiple number of user-specified parameters can be set by the user, e.g. the frequency f and the amplitude A of the electric pulse P, or the frequency f, the amplitude A and the pulse width w of the electric pulse P.

Optionally, the system 1 may further comprise a sensor sensing physiological data associated with the spinal cord injury or other tissue of the patient. By forwarding the sensed physiological data to the user interface 12 and/or the control unit 9 of the pulse processor 6, a parameter of the electric stimulation signal S can be adjusted, at least partially based on the sensed physiological data.

As an example, an electric signal at the distal end of an electrode can be measured by the sensor, so as to form a closed loop for controlling the electric signal to desired signal characteristics. As a further example, autonomous electrical activity in the target dorsal root ganglion DRG or adjacent tissue can be measured by the sensor, for monitoring local natural electric activity in the DRG or adjacent tissue of the patient, e.g. for tuning or matching the applied stimulation electric signal S, e.g. in shape, intensity or timing, to an electrical profile that is already present. As another example, physiological data can be measured by the sensor in more remote tissue, e.g. muscles that are governed by electric signals in the target dorsal root ganglion, e.g. for the purpose of providing an overall feedback to the stimulation signal.

FIG. 2 shows an electric stimulation signal S according to the invention for preventing and/or reducing muscle spasms and improving postural stability in a patient suffering from muscle spasm and postural instability. The shown stimulation signal S is a current signal. However, generally, the electric stimulation signal can be a current and/or a voltage signal. The shown electric stimulation signal S as a function a time parameter t has subsequent electric pulses P₁, P₂ having an amplitude Aₚ, a frequency f that is the reverse of the cycle time T defined between the center time instants t₁ and t₂ of the subsequent pulses P₁, P₂, and a pulse width w.

FIG. 3 shows a flow chart of a method according to the invention. The method is used for preventing and/or reducing muscle spasms and improving postural stability in a patient suffering from muscle spasm and postural instability. Generally, the muscle spasm and postural instability may be caused by a spinal cord injury or other leasions or diseases of the nervous system. The method 100 comprises a step of generating 110 an electric stimulation signal at an electrode positioned to stimulate a target dorsal root ganglion associated with the muscle spasm and postural instability, the stimulation signal stimulating the dorsal root ganglion with an electric pulse having at least one user-specified parameter value in a pre-specified parameter range, preferably having an amplitude between circa 0.01 mA and circa 2 mA, a repetition frequency between circa 1 Hz and circa 100 Hz, and a pulse width between circa 10 microseconds and circa 500 microseconds.

The step of generating an electric stimulation signal can be performed using dedicated hardware structures, such as FPGA and/or ASIC components. Otherwise, the method can at least partially be performed using a computer program product comprising instructions for causing a processor of a computer system to perform the above described steps. The step can in principle be performed on a single processor. However it is noted that at least a substep can be performed on a separate processor, e.g. a substep of amplifying the signal.

During a pilot study aimed at evoking motor response in patients with complete paraplegia (ASIA A/B) using DRG-stimulation, an incidental finding of suppressive quality of DRG-stimulation on the spasticity has been found in this patient group.

During the study, patients received a DRG-lead bilaterally on level L4 during a total of 5 days. On day 1 and 5, EMG-measurements were made during DRG-stimulation to objectify the evoked motor response. A total of 9 muscle groups were measured bilaterally, as well as the ECG signal for filtering purposes. Between day 1-5 the patients were sent home with a chronic low-frequency, low-amplitude stimulation protocol aimed at providing neuromodulation to test a potential shift in motor response thresholds on day 5. During their home-stimulation period patients were asked to fill in a diary with questions on pain sensation, motor response as well as other potentially interesting observations.

The incidental finding was based on two sources of information:
1) Of the total of n=4 patients measured so far, n=3 have reported in their diary, as well as during the evaluation interview, to have experienced less (‘frequency’) and less pronounced (‘severity’) problems with spasticity, as also objectified on a Visual Analogue Scale (VAS).
2) In one of our patients (pt. #03), the EMG measurements during day 1 and 5 were used to test the potential of DRG-stimulation to stop spontaneous spasticity, an example of which is illustrated referring to FIG. 4. By awaiting spasticity after postural change during these measurements, and directly responding by manually activating the DRG-stimulator on a low-frequency (<8 Hz) and low-amplitude (<500 μA) setting upon observing clinically visible spasticity, the spasticity was repeatedly observed to be suppressed to a clinically non-visible level.

In addition, of the total of n=4 patients measured so far, n=2 have reported in their diary, as well as during the evaluation interview, to have gained postural stability (‘I can sit better in my wheelchair’ and ‘I felt a new tightness around my back and stomach’).

FIG. 4A shows electromyography traces 200 of bilateral muscle groups of a patient. On the left hand side traces of the left muscle groups are shown, on the right hand side traces of the right muscle groups are shown. Here, the Paraspinal muscle is denoted by PS, the Vastus Lateralis muscle is denoted by VL, the Vastus Medialis is denoted by VM, the Iliopsoas is denoted by IL, the Rectus Femoris is denoted by RF, the Abductor Hallucis is denoted by AH, the Biceps Femoris is denoted by BF, the Gastrocnemius is denoted by GC, and the Tibialis Anterior is denoted by TA. In the overview of the EMG-traces of n=9 muscle groups bilaterally, n=7 of which seem to show spasticity in their traces (VL, VM, RF, AH, BF, GC, TA) on the right side. A dotted line 201 indicates an abrupt end of a spasm as based on the characteristics of the EMG-trace. The dotted line 201 also indicates a time instant when an electric stimulation signal starts. Parallel video-analysis confirmed a clinically visible end of spasticity at this same time-point. A left-hand side box 202 indicates a region of interest during spasm, prior to the dotted line 201, while a right-hand side box 203 indicates a region of interest after the dotted line 201, after the spasm.

FIG. 4B shows a frequency spectrum of a Vastus Lateralis electromyography trace prior to application of an electric stimulation signal. Similarly, FIG. 4C shows a frequency spectrum of the Vastus Lateralis electromyography trace after application of an electric stimulation signal. An analysis of the frequency spectrum of the signal during, FIG. 4B, and after spasm, FIG. 4C, reveals a shift from a relatively heterogeneous frequency signal, as expected from spasticity, to a relatively low-frequency only signal, after the spasm.

FIG. 4D shows the Vastus Lateralis electromyography trace as a function of time prior to application of an electric stimulation signal, and FIG. 4E shows the Vastus Lateralis electromyography trace as a function of time after application of an electric stimulation signal. Three second EMG-trace zoom-ins of the above regions of interest during the spasm, FIG. 4D, i.e. before the dotted line 201, as well as after the spasm, FIG. 4E, i.e. after the dotted line 201, again showing a difference in muscle reaction characteristics before and after spasm. Again, it is illustrated that a heterogeneous signal, shown in FIG. 4D, changes in a more regular, low-frequency (5-6 Hz) signal after the end of the spasm, as shown in FIG. 4E. Based on the abrupt end of spasm in all involved muscles, as well as the shift to a low frequency muscle signal after spasm, it is highly likely that the low-frequency, low-amplitude DRG-stimulation which was manually activated on the right side after observing clinically visible spasticity, was responsible for the suppression.

The invention is not restricted to the embodiments described above. It will be understood that many variants are possible.

These and other embodiments will be apparent for the person skilled in the art and are considered to fall within the scope of the invention as defined in the following claims. For the purpose of clarity and a concise description features are described herein as part of the same or separate embodiments. However, it will be appreciated that the scope of the invention may include embodiments having combinations of all or some of the features described.

1. A system for preventing and/or reducing muscle spasms and improving postural stability in a patient suffering from muscle spasm and postural instability, the system comprising:
an electrode configured to be positioned to stimulate a target dorsal root ganglion associated with the muscle spasm and postural instability;
a pulse generator provided with an output port connectable to the electrode, wherein the pulse generator includes a processor configured to generate an electric stimulation signal at the electrode while connected, the electric stimulation signal stimulating the dorsal root ganglion with an electric pulse while so positioned, and
wherein the system further comprises a user interface configured to set at least one user-specified parameter of the electric pulse.

2. The system according to claim 1, wherein the user interface is configured to set the at least one user-specified parameter of the electric pulse only to a respective value in a respective pre-specified range.

3. The system according to claim 1, wherein the at least one user-specified parameter includes a parameter representing an amplitude of the electric pulse ranging between circa 0.01 mA and circa 2 mA.

4. The system according to claim 1, wherein the at least one user-specified parameter includes a parameter representing a repetition frequency of the electric pulse ranging between circa 1 Hz and circa 100 Hz.

5. The system according to claim 1, wherein the at least one user-specified parameter includes a parameter representing a pulse width of the electric pulse ranging between circa 10 microseconds and circa 500 microseconds.

6. The system according to claim 4, wherein the at least one user-specified parameter also includes a parameter setting a sequence of high frequency electric pulses preceding, following or alternating electric pulses having a repetition frequency in the range between circa 1 Hz and circa 100 Hz, the high frequency electric pulses having a frequency above circa 100 Hz.

7. The system according to claim 6, wherein the sequence of high frequency electric pulses include high frequency pulses having a frequency that is significantly higher than circa 100 Hz, preferably circa 300 Hz, circa 500 Hz, circa 700 Hz or circa 10 kHz.

8. The system according to claim 1, comprising a multiple number of electrodes configured to be positioned to stimulate respective target dorsal root ganglions, wherein the pulse generator is provided with a multiple number of output ports connectable to the respective electrodes.

9. The system according to claim 8, wherein a set of electrodes from the multiple number of electrodes is bundled in a string of electrodes.

10. The system according to claim 1, wherein the processor and the control unit are implantable.

11. The system according to claim 1, further comprising a sensor sensing physiological data associated with the spinal cord injury, wherein the user interface is further configured to adjust a parameter of the electric pulse based on sensed physiological data.

12. A method of preventing and/or reducing muscle spasms and improving postural stability in a patient suffering from muscle spasm and postural instability, the method comprising generating an electric stimulation signal at an electrode positioned to stimulate a target dorsal root ganglion associated with the muscle spasm and postural instability, the stimulation signal stimulating the dorsal root ganglion with an electric pulse having at least one user-specified parameter value in a pre-specified parameter range.

13. The method according to claim 12, wherein the electric pulse has an amplitude between circa 0.01 mA and circa 2 mA, a repetition frequency between circa 1 Hz and circa 100 Hz, and a pulse width between circa 10 microseconds and circa 500 microseconds.

14. The method according to claim 12, wherein the electrode is positioned at the dorsal root ganglion of L1, L2, L3, L4, L5, S1, S2, S3, S4, S5, Thoracic or Cervical levels.

15. A computer program product for preventing and/or reducing muscle spasms and improving postural stability in a patient suffering from muscle spasm and postural instability the computer program product comprising computer readable code for causing a processor to perform a step of generating an electric stimulation signal at an electrode positioned to stimulate a target dorsal root ganglion associated with the muscle spasm and postural instability, the stimulation signal stimulating the dorsal root ganglion with an electric pulse having at least one user-specified parameter value in a pre-specified parameter range.

16. (canceled)
